(19) United States
(12) Patent Application Publication
(10) Pub. No.: US 2024/0017006 A1
(43) Pub. Date: Jan. 18, 2024

(54) APPARATUSES AND METHODS FOR PAUSING AN INFUSION PUMP DURING A DISPENSE STROKE TO IMPROVE OCCLUSION SENSING

(71) Applicant: Becton, Dickinson and Company, Franklin Lakes, NJ (US)

(72) Inventors: Scott STEWART, Pittsburgh, PA (US); Scott COREY, Hydes, MD (US); Kun LI, Boyds, MD (US)

(73) Assignee: Becton, Dickinson and Company, Franklin Lakes, NJ (US)

(21) Appl. No.: 18/254,677

(22) PCT Filed: Dec. 9, 2021
(86) PCT No.: PCT/US21/62546

§ 371 Date: May 26, 2023

Related U.S. Application Data
(60) Provisional application No. 63/125,497, filed on Dec. 15, 2020.

(51) Int. Cl. A61M 5/168; A61M 5/145

(52) U.S. Cl. CPC A61M 5/16831; A61M 5/1452; A61M 2005/16863

(57) ABSTRACT

Devices and methods detect and mitigate pre-occlusion leak pressures within a dispense stroke of an infusion device. An infusion pump obtains pump measurements indicative of pressure, and controls its pump mechanism to pause when the pump measurements satisfy a pre-occlusion pressure threshold. Additional pump measurements are obtained during the pause and pressure-related conditions can abate during the pause. The dispense stroke is resumed by the infusion pump when the pump measurements no longer satisfy pre-occlusion pressure criteria, and can be paused again. The number and frequency of pauses, analysis of pump measurements, and resumption of a dispense stroke can be predetermined, or dynamically determined. Pauses can also be preset regardless of current pressure conditions. Pausing the pump to increase the samples or sampling rate of a measured parameter indicative of pressure (e.g., pump motor current) provides a much higher resolution for detecting an occlusion.

### BACKGROUND

### Field

Illustrative embodiments relate generally to detecting and mitigating pre- occlusion leak pressures within a dispense stroke of an infusion device.

### Description of Related Art

Anomalies or dysfunctions such as leaks, occlusions or presence of air bubbles in a fluid path can occur in an infusion pump and are not necessarily noticeable to the user. Detection of a dysfunction such as a partial or total occlusion along a fluid path in an infusion pump can be desirable to maintain accurately controlled medication delivery and to advise the user to discontinue use of a malfunctioning infusion device. A typical solution for occlusion detection is to place a pressure sensor in the infusion pump system and report occlusion when the pressure is above a certain threshold. Adding a pressure sensor, however, increases the complexity of the system (e.g., increases mechanical, electrical, and/or software complexity), increases system power consumption, and increases the cost of the infusion pump.

For medical devices such as a wearable medication delivery pump, where some or all of the components are disposable for ease of use and cost effectiveness, adding another component such as a pressure sensor and related increased cost and complexity to the medical device is undesirable. A need therefore exists for accurate occlusion detection without adding infusion pump components and thereby increasing infusion pump complexity and cost.

In addition, pressure sensing systems in pumps may be unable to detect a leak pressure condition quickly enough to avoid failure because of low compliance of the downstream fluid path, which can result in very rapid increases in pressure.

### SUMMARY

The above and other problems are overcome, and additional advantages are realized, by illustrative embodiments.

In accordance with aspects of illustrative embodiments, an infusion device is provided that comprises: a pump comprising a chamber of fluid, and a pumping mechanism configured to control dispensing of a volume of fluid from the chamber during a dispense operation; a pump measurement device configured to generate pump measurements indicative of pressure; and a processing device configured to analyze one or more of the pump measurements obtained during a portion of the dispense operation, and pause the pump mechanism during the dispense operation when one or more of the pump measurements during the portion of the dispense operation satisfies a designated metric related to a designated pre-occlusion pressure.

In accordance with aspects of illustrative embodiments, the processing device is configured to control the pump mechanism to resume the dispense operation. For example, the processing device can be configured to obtain another pump measurement during the dispense operation resumed after the pause, and to pause the pump mechanism again when the pump measurement satisfies a designated metric related to a designated pre-occlusion pressure. As a further example, the processing device is configured to control the pump mechanism to resume the dispense operation again. As another example, the number of times the processing device pauses the dispense operation and resumes the dispense operation can be preconfigured, or dynamically determined, based on criteria chosen from pump motor current, pump motor voltage, encoder count, pump motor drive count, pump motor drive time, dispense operation energy, volume of the chamber, type of fluid in the chamber, and ambient air pressure.

In accordance with aspects of illustrative embodiments, the frequency with which the processing device pauses and resumes the dispense operation can be constant or vary throughout the dispense operation or within designated portions of the dispense operation.

In accordance with aspects of illustrative embodiments, the processing device can be configured to perform a feedback loop of obtaining pump measurements and slowing down or speeding up the dispense operation based on the pump measurements.

In accordance with aspects of illustrative embodiments, the pump measurement is motor current, and the pump measurement device comprises a current sensing device configured to detect motor current of the pump during the dispense operation.

In accordance with aspects of illustrative embodiments, the pump is chosen from a positive displacement pump, and a syringe-style pump.

In accordance with aspects of illustrative embodiments, the designated metric relates to a designated pre-occlusion pressure is chosen from a range of measurements corresponding to pressures above normal pump operating pressures, and below a minimum leak pressure, and different from transient pressures related to pump start up or pump operation state change.

In accordance with aspects of illustrative embodiments, the processing device is configured to obtain at least an additional pump measurement during the pause, and controls the pump mechanism to resume the dispense operation when the additional pump measurement corresponds to a normal pump operating pressure and fails to satisfy the designated metric related to a designated pre-occlusion pressure.

Additional and/or other aspects and advantages of illustrative embodiments will be set forth in the description that follows, or will be apparent from the description, or may be learned by practice of the illustrative embodiments. The illustrative embodiments may comprise apparatuses and methods for operating same having one or more of the above aspects, and/or one or more of the features and combinations thereof. The illustrative embodiments may comprise one or more of the features and/or combinations of the above aspects as recited, for example, in the attached claims

### BRIEF DESCRIPTION OF THE DRAWINGS

The above and/or other aspects and advantages of the illustrative embodiments will be more readily appreciated from the following detailed description, taken in conjunction with the accompanying drawings, of which:

FIGS. 1 and 2 are partial, perspective views of example pump components in an example medication delivery device that operates in accordance with an occlusion detection algorithm in accordance with an illustrative embodiment of the present invention;

FIGS. 3A and 3B are perspective views of pump components of FIGS. 1 and 2 in an example medication delivery device arranged, respectively, in accordance with a ready to dispense stage of operation and a ready to aspirate stage of operation;

FIG. 3C is a perspective view of components in an example medication delivery device comprising example pump components of FIGS. 1 and 2 and associated electronic circuits on a printed circuit board;

FIG. 4A is a block diagram of components in an example medication delivery device;

FIG. 4B is a schematic diagram of a medication delivery device pump motor having a current sensor in accordance with an illustrative embodiment of the present invention;

FIG. 5 depicts pump measurement data from an example delivery device indicating motor current during a dispense stroke before and after occlusion;

FIG. 6 depicts pump measurement data from an example delivery device indicating pressure changes relative to respective pump strokes over time;

FIG. 7 depicts pump measurement data from an example delivery device correlated to pressure over time;

FIG. 8 is a flow chart of illustrative operations of an example medication delivery device performing an occlusion detection algorithm in accordance with an illustrative embodiment of the present invention;

FIG. 9 is a perspective view of an example wearable, syringe-style fluid delivery device employing an occlusion detection algorithm in accordance with an example embodiment; and

FIGS. 10A, 10B, 10C and 10D are, respectively, a partial top view, a perspective view, a side view, and a top view of the example fluid delivery device of FIG. 9 with the cover removed.

Throughout the drawing figures, like reference numbers will be understood to refer to like elements, features and structures.

### DETAILED DESCRIPTION OF ILLUSTRATIVE EMBODIMENTS

Reference will now be made in detail to example embodiments of the present disclosure, which are illustrated in the accompanying drawings. The example embodiments described herein exemplify, but do not limit, the claimed invention and present disclosure by referring to the drawings.

Occlusion in a fluid pump can result from restricted flow or pathway constriction such as a pinched catheter or tissue occlusion in a fluid delivery device such as an infusion pump for medication. It is important to measure pump pressure changes from an occlusion or other pump condition (e.g., empty reservoir) for early detection of pump malfunction and possible fluid delivery inaccuracies resulting therefrom.

Different methods of detecting pump occlusions involve using a force sensor in a fluid pathway, or measuring a pump motor parameter such as motor current, motor voltage, encoder count, motor drive count, delivery pulse energy, motor drive time, and so on. For example, current sensing is generally considered to be a reliable method of detecting occlusions in a fluid path of a fluid delivery device because motor current can be indirectly correlated to pump pressure. An occlusion causes a decrease in fluid flow, which causes increased pressure. Increased pressure causes increased torque demand by the pump motor, and increased torque demand by the pump motor draws more current.

In related pump systems described in in commonly owned WO 2019/156852 and W02019/156848, the current of single strokes are measured and aspects of these strokes are normalized and then compared to thresholds. If specific characteristics of a stroke meet algorithm thresholds, the fluid delivery device triggers an occlusion notification. One issue with this strategy is that the fluid delivery device collects only one data point per stroke (e.g., when it compares ranges of stroke data to a threshold). If the downstream path of the fluid delivery device is characterized by low compliance, downstream fluid path pressure can increase very rapidly (e.g., on the order of 30+psi within one stroke for some fluid delivery devices). This is problematic because, in one stroke, fluid pressure can go from a level at which the fluid delivery device should not detect occlusion (e.g., 18 psi for some devices), to above a designated leak pressure of the device (e.g., 45 psi) without detection, and potentially cause a failure.

In accordance with advantageous aspects of example embodiments of the present disclosure, an occlusion sensing algorithm provides more data points within a fluid delivery device pump stroke to measure pressure and mitigate against leak pressures without significantly modifying the output or dispense operations of the fluid delivery device. The occlusion sensing algorithm controls a fluid delivery device to obtain a measurement of a device parameter that is indicative of fluid pressure during a stroke (e.g., a dispense stroke), and to control a pump mechanism in the fluid delivery device to pause during the stroke when the measurement satisfies a criteria corresponding to designated pre-occlusion threshold (T_{PRE-OCC}). If the fluid delivery device measurement indicates moderate pressure during a dispense stroke, even if that measurement does not meet criteria designated as an occlusion (T_{OCC}), the fluid delivery device does not complete the dispense stroke right away. Instead, during a pause within the stroke, the fluid delivery device is controlled to obtain one or more measurement(s) or sample(s) of data that can correlate to device pressure during the time when pressure is increasing. If the additional measurement(s) does not satisfy a designated pre-occlusion threshold (T_{PRE-OCC}), then the algorithm controls the pump mechanism to resume the stroke.

The number of times that the occlusion sensing algorithm pauses, obtains measurements, and resumes a stroke in this manner can depend on optimizing between collecting measurement data correlating to pressure conditions in the fluid delivery device and slowing down the time to deliver an entire intended dose, which may consist of one or multiple strokes. Accordingly, the occlusion sensing algorithm in accordance with example embodiments provides 1) more opportunities to detect a pressure rise before it gets too high and to cause failure, and 2) more opportunities to reaffirm the original marginal measurement and thereby reduce noise. In accordance with an example embodiment, the occlusion sensing algorithm does not pause on every stroke, but only on those strokes during which the measurement of the device parameter relied upon to indicate fluid pressure is deemed to be unusual or otherwise not satisfy a designated criteria such as a lower pre-occlusion threshold.

Example embodiments of the present disclosure are illustrated and described wherein motor current is the parameter to be measured as an indication of pressure. It is to be understood, however, that one or more different methods can be used for detecting occlusions. For example, a fluid delivery device can be provided with a pressure or force sensor along a fluid path. Alternatively or in addition to adding a pressure sensor, a different pump motor parameter that is indicative of pressure can be measured such as, but not limited to, motor voltage, motor drive time, motor coast time, delivery pulse energy, motor drive count, motor coast count, and delta encoder count, among other parameters.

The example embodiments of occlusion sensing algorithm are particularly useful with respect to positive displacement pumps. A positive displacement pump is understood to be a type of pump that works on the principle of filling a chamber (e.g., with liquid medication from a reservoir) in one stage and then emptying the fluid from the chamber (e.g., to a delivery device such as a cannula deployed in a patient) in another stage. For example, a reciprocating plunger-type pump or a rotational metering-type pump can be used. In either case, a piston or plunger is retracted from a chamber to aspirate or draw medication into the chamber and allow the chamber to fill with a volume of medication (e.g., from a reservoir or cartridge of medication into an inlet port). The piston or plunger is then re-inserted into the chamber to dispense or discharge a volume of the medication from the chamber (e.g., via an outlet port) to a fluid pathway extending between the pump and a cannula in the patient. Alternatively, example embodiments of occlusion sensing algorithm can be extended for infusion syringe-style pumps as well. While the positive displacement pump iteratively fills and dispenses a chamber, a syringe pump continually dispenses the chamber also referred as reservoir. The dosing regimen could be discrete, segmented such that smaller doses (similar to ones found in positive displacement pumps) can be analyzed within the larger reservoir volume delivery. Through this discretization, individual doses and their parameters can be analyzed just as described herein for positive displacement pumps.

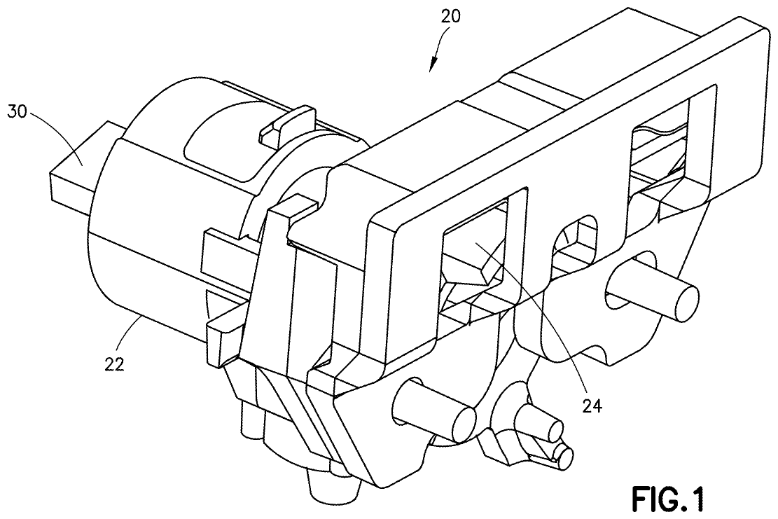

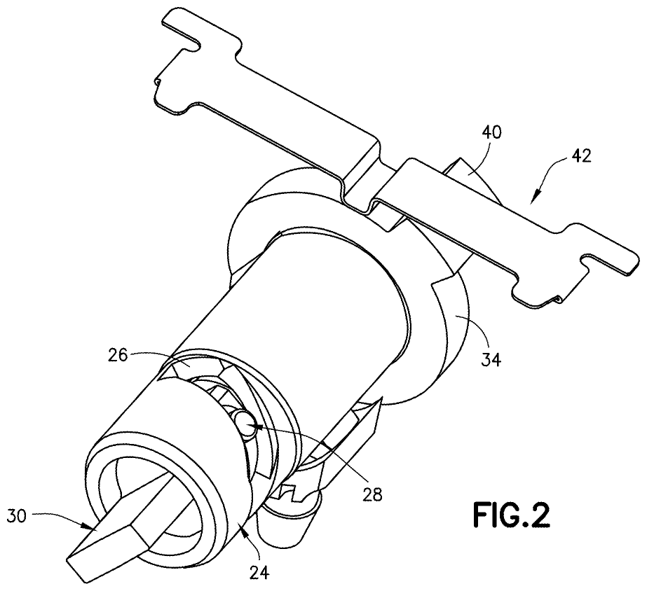

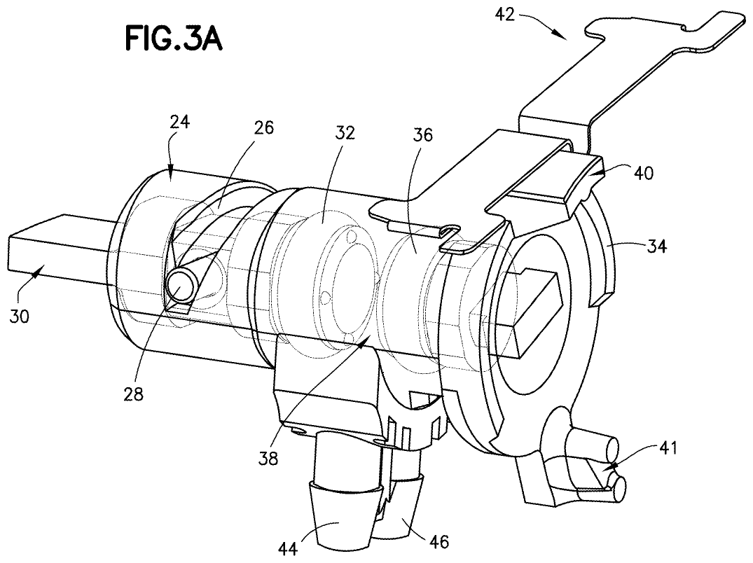

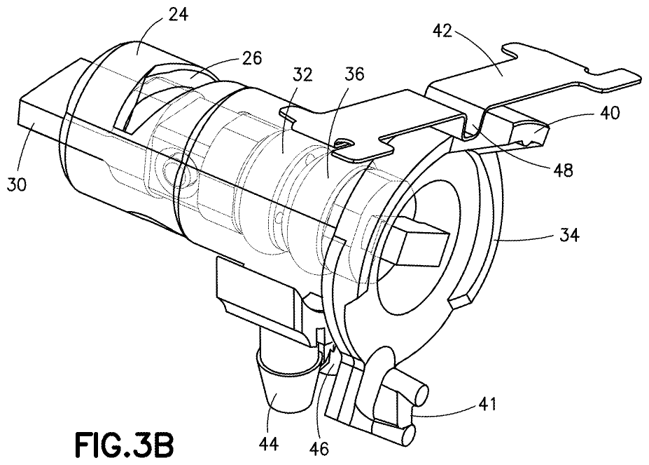

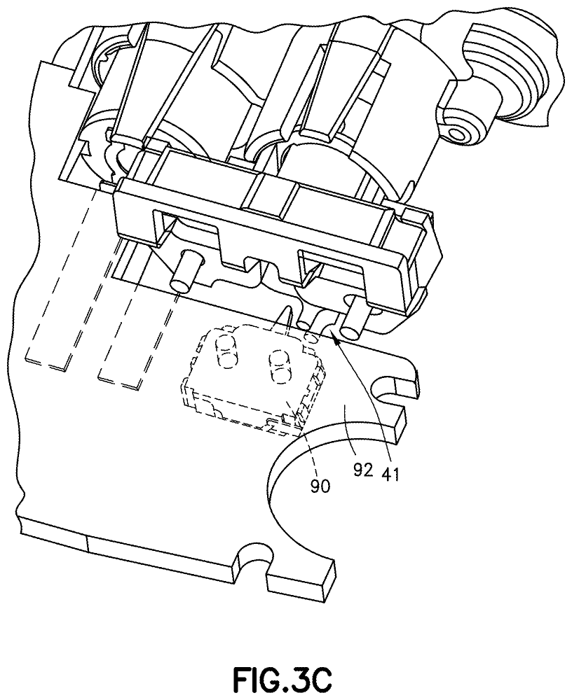

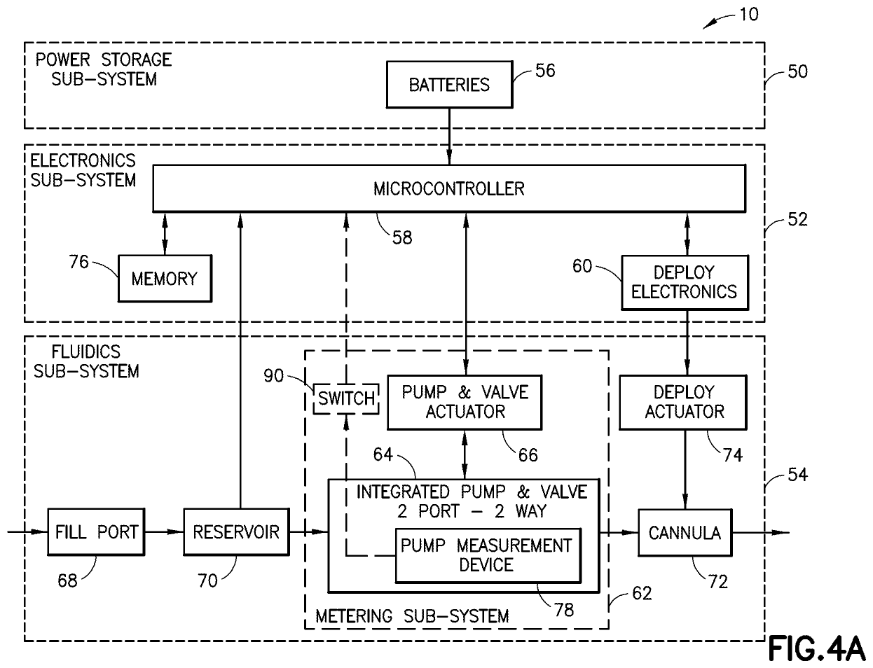

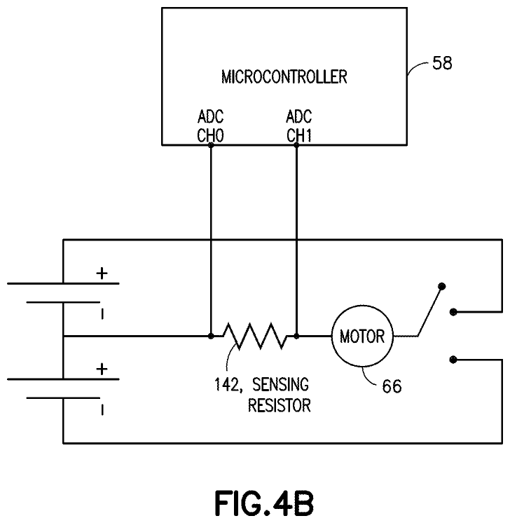

For illustrative purposes, reference is made to an example rotational metering-type pump described in commonly owned WO 2015/157174, the content of which is incorporated herein by reference in its entirety. With reference to FIGS. 1, 2, 3A, 3B and 3C, an example infusion pump (e.g., a wearable medication delivery device such as an insulin patch pump) comprises a pump assembly 20 which can be connected to a DC motor and gearbox assembly (not shown) to rotate a sleeve 24 in a pump manifold 22. A helical groove 26 is provided on the sleeve. A coupling pin 28 connected to a piston 30 translates along the helical groove to guide the retraction and insertion of the piston 30 within the sleeve 24, respectively, as the sleeve 24 rotates in one direction and then rotates in the opposite direction. The sleeve has an end plug 34. Two seals 32, 36 on the respective ends of the piston and end plug that are interior to the sleeve 24 define a cavity or chamber 38 when the piston 30 is retracted, as depicted in FIG. 3A, following an aspirate stroke and therefore ready to dispense. The volume of the chamber 38 therefore changes depending on the degree of retraction of the piston 30. The volume of the chamber 38 is negligible or essentially zero when the piston 30 is fully inserted and the seals 32, 36 are substantially in contact with each other following a dispense stroke, as depicted in FIG. 3B, and therefore ready to aspirate. Two ports 44, 46 are provided relative to the pump manifold 22, including an inlet port 44 through which medication can flow from a reservoir 70 (FIG. 4A) for the pump 64 (FIG. 4A), and an outlet port 46 through which the medication that has been drawn into the chamber 38 (e.g., by retraction of the piston 30 during an aspirate stage of operation) can be dispensed from the chamber 38 to, for example, a fluid path to a cannula 72 (FIG. 4A) in the patient by re-insertion of the piston 30 into the chamber 38.

With continued reference to FIGS. 1, 2, 3A, 3B and 3C, the sleeve 24 can be provided with an aperture (not shown) that aligns with the outlet port 46 or the inlet port 44 (i.e., depending on the degree of rotation of the sleeve 24 and therefore the degree of translation of the piston 30) to permit the medication in the chamber 38 to flow through the corresponding one of the ports 44, 46. A pump measurement device 78 (FIG. 4A) such as a sleeve rotational limit switch can be provided which has, for example, an interlock 42 and one or more detents 40 on the sleeve 24 or its end plug 34 that cooperate with the interlock 42. The interlock 42 can be mounted to the manifold 22 at each end thereof. The detent 40 at the end face of sleeve 24 is adjacent to a bump 48 of the interlock 42 when the pump 64 is in a first position whereby a side hole in the sleeve 24 is aligned with the inlet port 44 to receive fluid from the reservoir 70 into the chamber 38. Under certain conditions, such as back pressure, it is possible that friction between the piston 30 and the sleeve 24 is sufficient to cause the sleeve 24 to rotate before the piston 30 and coupling pin 28 reach either end of the helical groove 26. This could result in an incomplete volume of liquid being pumped per stroke. In order to prevent this situation, the interlock 42 prevents the sleeve 24 from rotating until the torque passes a predetermined threshold, as shown in FIG. 3A. This ensures that piston 30 fully rotates within the sleeve until the coupling pin reaches the end of the helical groove 26. Once the coupling pin 28 hits the end of the helical groove 26, further movement by the DC motor and gearbox assembly or other type of pump and valve actuator 66 (FIG. 4A) increases torque on the sleeve 24 beyond the threshold, causing the interlock 42 to flex and permit the detent 40 to pass by the bump 48. At the completion of rotation of the sleeve 24 such that its side hole is oriented with the cannula 72 or outlet port 46, the detent 40 moves past the bump 48 in the interlock 42, as shown in FIG. 3B. Another sleeve feature 41 can be provided to engage an electrical switch (e.g., an end-stop switch 90 provided on a printed circuit board 92 and disposed relative to the sleeve and/or end plug 34 to cooperate with the pump measurement device 78 as shown in FIG. 3C).

FIG. 4A is an illustrative system diagram that illustrates example components in an example medication delivery device 10 having an infusion pump such as the pump of FIGS. 1, 2, 3A, 3B and 3C. The medication delivery device 10 can include an electronics sub-system 52 for controlling operations of components in a fluidics sub-system 54 such as the pump 64 and an insertion mechanism 74 for deploying a cannula 72 for insertion into an infusion site on a patient's skin. A power storage sub-system 50 can include batteries 56, for example, for providing power to components in the electronics and fluidics sub-systems 52 and 54. The fluidics sub-system 54 can comprise, for example, an optional fill port 68 for filling a reservoir 70 (e.g., with medication), although the medication delivery device 10 can be optionally shipped from a manufacture having its reservoir already filled. The fluidics sub-system 54 also has a metering sub-system 62 comprising the pump 64 and a pump actuator 66. As described above, the pump 64 can have two ports 44, 46 and related valve sub-assembly that controls when fluid enters and leaves a pump chamber 38 via the respective ports 44, 46. One of the ports is an inlet port 44 through which fluid such as liquid medication flows from the reservoir 70 into the pump 64 as the result of a pump intake or pull stroke on a pump plunger or piston 30, for example. The other port is an outlet port 46 through which the fluid leaves the pump's chamber 38 and flows toward a cannula 72 for administration to a patient pump as the result of a pump discharge or push stroke on the pump plunger or piston 30. The pump actuator 66 can be a DC motor and gearbox assembly or other pump driving mechanism for controlling the plunger or piston 30 and other related pump parts such as a sleeve 24 that may rotate relative to the translational movement of the pump piston 30. The microcontroller 58 can be provided with an integrated or separate memory device having computer software instructions to actuate, for example, rotation of the sleeve 24 in a selected direction, translational or axial movement of a piston 30 in the sleeve 24 for an aspirate or dispense stroke, and optionally the rotation of the sleeve 24 and piston together during a valve state change as described in the above-referenced WO 2015/157174. As described below, an occlusion detection algorithm in accordance with illustrative embodiment can be provided to the microcontroller 58 to monitor pump parameter measurements and detect when an occlusion condition occurs relative to the infusion pump.

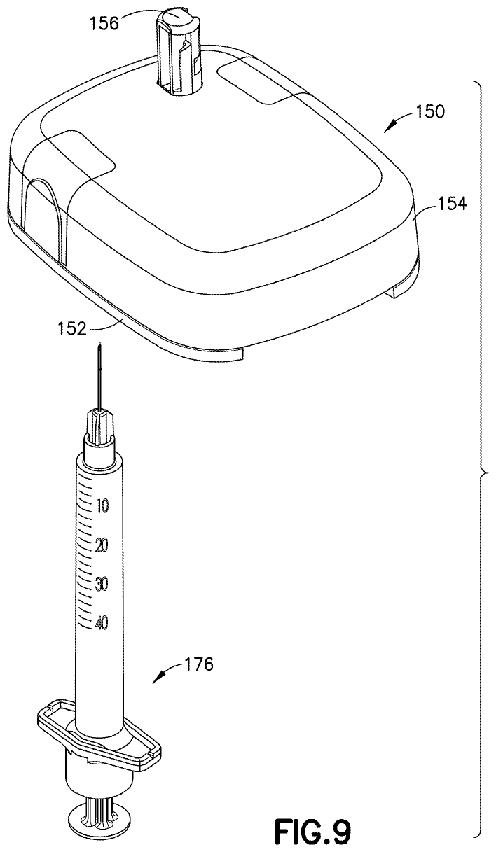

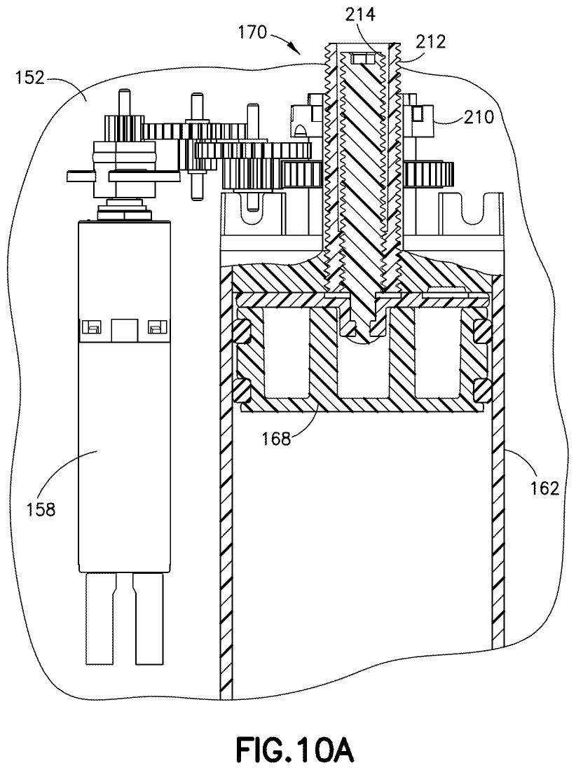

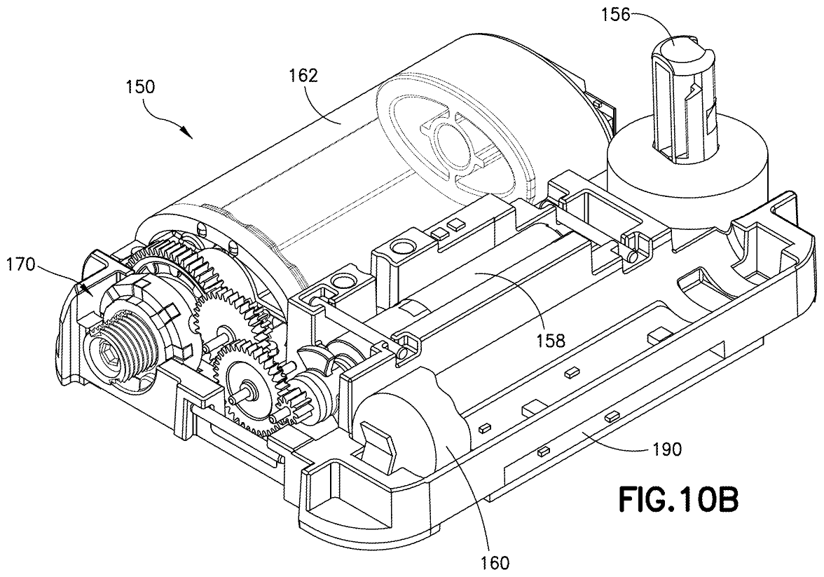

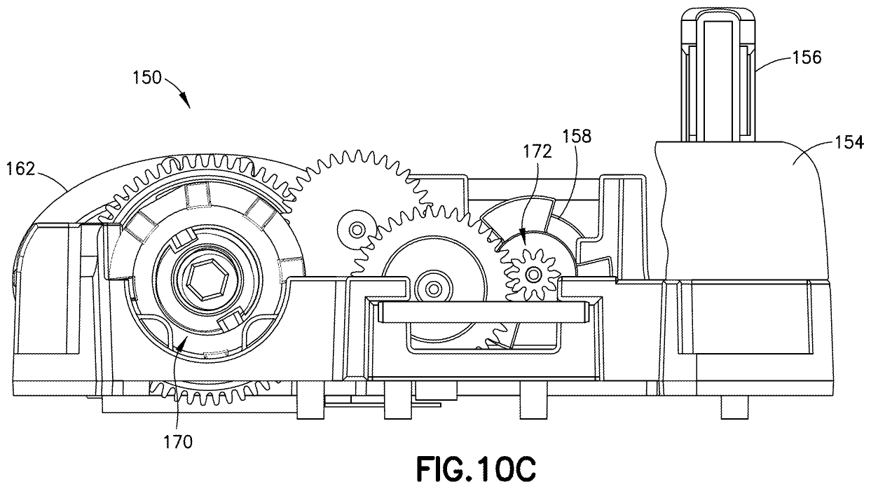

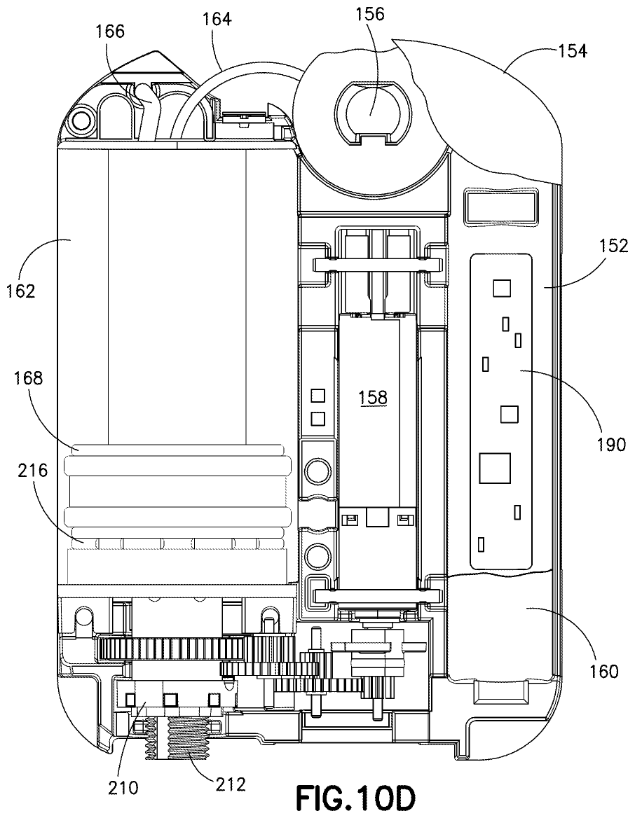

As stated above, example embodiments of occlusion sensing algorithm can be extended for an infusion syringe-style or piston-style pump. FIGS. 9 and 10A-10D illustrate an example syringe-style pump 150 described in co-pending, co-owned PCT application that claims the benefit of U.S. provisional patent application Ser. No. 63/125,508, filed Dec. 2020. The syringe-style pump 150 is shown in FIG. 9 with a baseplate 152 and cover 154. A user can insert the needle of a filled syringe 176 into a fill port (not shown) provided in the baseplate 152 that has an inlet fluid path 166 from the fill port to the reservoir 162 as shown in FIG. 10D. The baseplate 152 supports the insertion mechanism 156, a motor 158, a power source such as a battery 160, a control board 190, and a reservoir 162 or container for storing a fluid to be delivered to a user via an outlet fluid path 164 from and outlet port of reservoir to the insertion mechanism 156. The reservoir 162 can also have an inlet port connected via the inlet fluid path 166 to a fill port (e.g., provided in the baseplate 12). The reservoir 162 contains a plunger 168 having a stopper assembly. The proximal end of the reservoir 162 is also provided with a plunger driver assembly 170. The plunger driver assembly 170 can be a telescoping, simultaneously counter-rotating sleeve screw 212 and center screw 214, a gear anchor 174, a nut 210 that is rotated via a gear train 172 connected to the motor 158 and gearbox 184. It is to be understood that the plunger driver assembly 170 can comprise different components for pushing and extracting the plunger 168 within the reservoir 162. A controller 192 syringe-style pump 150 can be programmed or otherwise configured to operate the plunger driver assembly 170 to dispense fluid, and to perform the occlusion detection algorithm in accordance with the example embodiments of the technical solution described herein.

As stated above, a typical solution for occlusion detection is to place an additional pressure sensor in the pump control system and report occlusion when the pressure is above a certain threshold. Adding a pressure sensor, however, has the drawbacks of increasing the complexity of the system (e.g., mechanical, electrical, and/or software complexity), increasing system power consumption, and/or increasing pump cost. These drawbacks can be particularly disadvantageous to a wearable pump design wherein all or part of the pump is intended to be disposable once the reservoir 70 is emptied or the pump 64 has been used a selected amount of time and/or used to deliver a selected amount of medication.

In accordance with illustrative embodiments, occlusion detection is accomplished without an additional component such as an occlusion sensor deployed upstream or downstream of the pump 64. A microcontroller 58 or other processing device for controlling pump operation can be further controlled to determine when a motor parameter measurement is outside a designated range of normal operating conditions and therefore indicates an occlusion, and to generate an indication of detected occlusion. The pump 64 and/or the entire medication delivery device 10 can therefore, in turn, be replaced or repaired, thereby ensuring that the patient is receiving the full intended dosage that is provided under normal operating conditions.

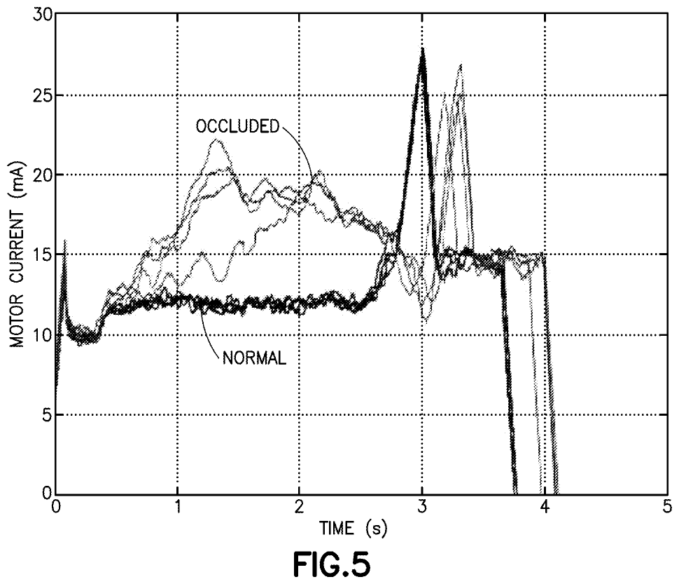

As stated above, example embodiments of the present disclosure are illustrated and described wherein motor current is the parameter to be measured as an indication of pressure. FIG. 5 illustrates motor current during a normal, non-occluded stroke, and motor current during an occluded pump stroke.

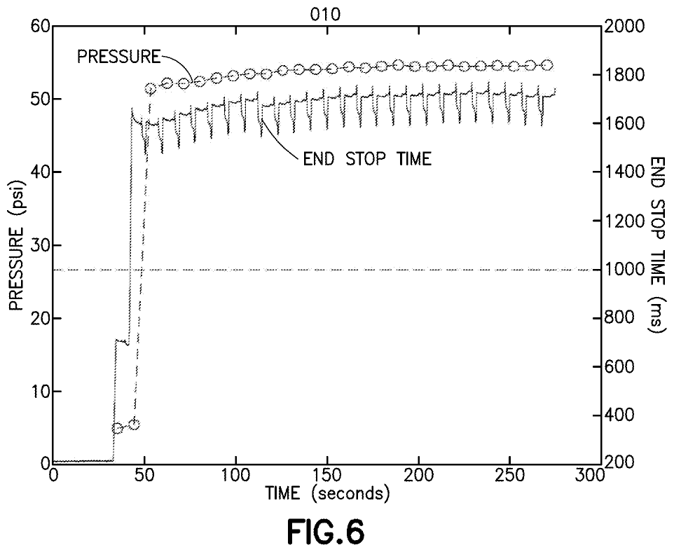

As stated above, fluid delivery devices can be characterized as having low compliance in their downstream fluid path due, for example, to an essentially incompressible fluid being passed through small hard plastic volume such as the fluid chamber outlet and cannula connection. Thus, pressure can increase very rapidly and potentially reach leak pressure within the duration of a single dispense stroke without detection or any opportunity for mitigation of pressure before a failure occurs. This rapid increase in pressure is illustrated in FIG. 6. FIG. 6 shows a series of strokes and corresponding pressures. If a fluid delivery device were programmed to detect occlusion using a pressure threshold of around 25 psi, then the first stroke with pressure at 18 psi (e.g., due to back pressure) should not be flagged. If, however, the fluid delivery device leaks at 45 psi, then it may be too late to flag an occlusion by the second stroke as illustrated in FIG. 6. FIG. 6 therefore illustrates the importance of measuring more than once per stroke using the occlusion detection algorithm in accordance with example embodiment described herein so that the probability of failing to detect this type of event decreases significantly.

FIG. 6 also illustrates some considerations for selecting at least one or more pre-occlusion threshold(s) and an occlusion threshold, or designated range of measured motor currents and correlated pressures that are not to be flagged as an occlusion from within the normal operational pressures of the fluid delivery device but are flagged as a sufficient pressure to pause the motor and take more motor current measurements in accordance with the occlusion detection algorithm. For example, the occlusion detection algorithm can be configured to not flag pressures below about 20 psi for pausing the pump motor during the dispense stroke since these are typical pressures for a given type of pump motor during a dispense stroke of a non-occluded pump due to back pressure. Further, the occlusion detection algorithm can be configured to flag above 45 psi as a leak pressure threshold due to occlusion (T_{OCC}). Thus, measured current correlating to pressures within a pre-occlusion threshold (T_{PRE-OCC}) range of 20-40 psi can be flagged by the microcontroller or other processor to pause the pump motor and take at least one additional measurement of motor current or other parameter indicative of pressure before continuing the dispense stroke in accordance with the occlusion detection algorithm. It is to be understood that these values for T_{OCC} and T_{PRE-OCC} range differ among different types of pumps and can be determined for a given type of pump empirically through testing.

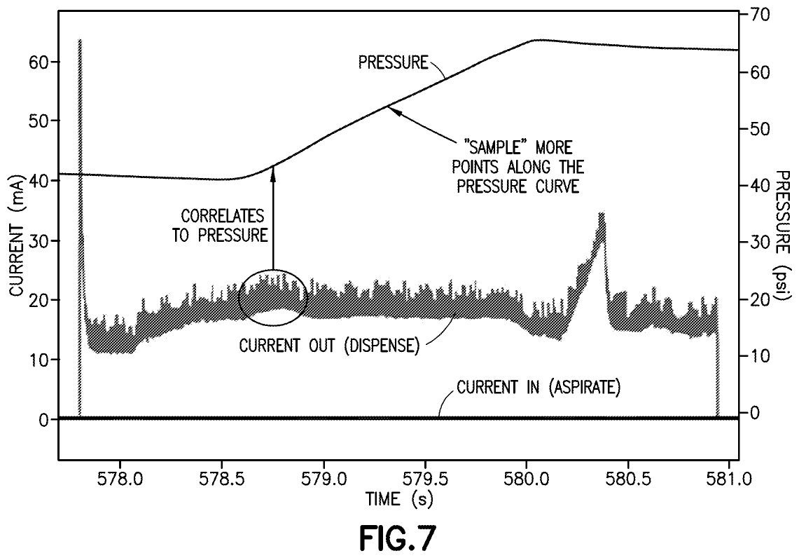

FIG. 7 illustrates an example pressure curve derived from motor current measurements for a given pump. The pressure curve is amplified to more clearly illustrate where one or more samples of current measurements can be advantageously taken along a portion of the pressure curve after pausing the pump motor when a pressure is detected that is within a T_{PRE-OCC} pressure range (i.e., higher than normal pressures when no occlusion is occurring and lower than a leak pressure threshold T_{OCC}). The frequency with which these measurements are made, the number of these measurements, and/or where within the pressure curve or pump stroke these measurements are made can be pre-determined or dynamically determined. For example, the number and/or frequency of measurements and the number of pause operations can increase with increasing value of the parameter within the T_{PRE-OCC} pressure range, or change based on the amount of dispense stroke that remains.

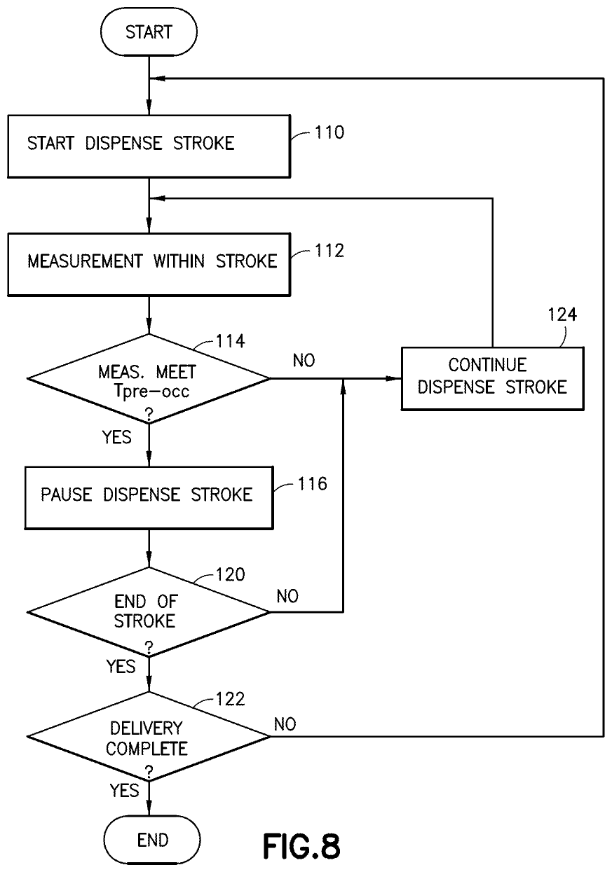

An occlusion detection algorithm in accordance with example embodiments will now be described with reference to FIG. 8. A measurement of a parameter indicative of pressure in a medication delivery device is measured (block 112) at some point after commencement of a dispense stroke of a pump cycle (block 110). The initial point of measurement following commencement of a dispense stroke can be pre-determined, or dynamically determined based on different factors described below. If the pump measurement indicative of pressure in the pump satisfies a pre-occlusion threshold T_{PRE-OCC} (block 114), then the microcontroller 58 or other pump processing device operating in accordance with the occlusion detection algorithm pauses the pump motor during the dispense stroke (block 116). As stated above, pausing the pump motor decreases the likelihood that a medication delivery device will experience undetected leak pressure within a dispense stroke. Optionally, another parameter measurement can be obtained during the pause.

If the end of the dispense stroke is not reached (block 120), then the microcontroller 58 continues the dispense stroke (block 124) in accordance with the occlusion detection algorithm. The microcontroller 58 is configured to obtain another measurement of a parameter indicative of pressure such as motor current (block 112). The occlusion detection algorithm can be configured to determine when (i.e., during the remainder of the dispense stroke) the measurements are made. For example, the occlusion detection algorithm can be configured to sample the measured parameter at a selected time interval after the dispense stroke is resumed, or a selected time or frequency depending on value of the measured parameter or the amount of dispense stroke that remains to be executed. In accordance with another example embodiment, the pump motor can be slowed down via pulse width modulation (PWM) or some other control means to enable the microcontroller 58 to implement a continuous feedback loop of measuring a parameter indicative of pressure and then slowing down or speeding up the dispense stroke based on that measurement.

If a measurement in block 112 satisfies a pre-occlusion threshold T_{PRE-OCC} (block 114), then the microcontroller 58 pauses the pump motor again. If the end of the dispense stroke is reached (block 120) and delivery is complete (block 122), then the process ends in FIG. 8 until the next dispense stroke by that pump or otherwise the pump is shut-down.

With continued reference to FIG. 8, the additional measurement in block 118 is evaluated to see if it satisfies a pre-occlusion threshold T_{PRE-OCC} (block 120). The pre-occlusion threshold TPRE-OCC in block 120 can be the same as, or different from, the T_{PRE-OCC} used in block 114. The occlusion detection algorithm can be configured regarding how many T_{PRE-OCC} measurements are made within a dispense stroke. For example, if a second measurement (block 118) continues to indicate an elevated pressure (e.g., satisfies a pre-occlusion threshold T_{PRE-OCC} per block 120), then the microcontroller 58 can be programmed to end the pump cycle and terminate pump operation, or obtain an additional measurement(s) (block 122). The additional measurement(s) and continued pause of the pump motor can allow for some pressure condition(s) to abate, and can allow additional measurements to avoid a rapid rise to leak pressure that is not detected in time for mitigation or adequate warning and pump shut-down to avoid dosing inaccuracy.

In accordance with another example embodiment, the occlusion detection algorithm can be configured to pause the pump during a dispense stroke after the stroke is halfway completed, or partially completed by other amount, instead of pausing the pump based on a measurement satisfying a criteria such as a pre-occlusion threshold T_{PRE-OCC}. For example, the pump can be configured by the occlusion detection algorithm to push a piston-style or syringe-style pump halfway through a dispense stroke, pause the piston movement, resume pushing the piston a partial stroke amount, analyze the motor current signal for determine if a pre-occlusion threshold TpRE-occ criteria is satisfied, and repeat several times within a dispense stoke to obtain multiple readings per stroke and avoid reaching leak pressure. Thus, if the pump catheter is pinched (e.g., tissue occlusion and restricted flow), then the occlusion detection algorithm allows the pump to slow down and the pressure to dissipate, thereby mitigating partial occlusion or other high pressure anomaly. The pump can be configured in accordance with the occlusion detection algorithm to look at a designated number of segments of a dispense stroke or selected zones in a dispense stroke and analyze a measured parameter to determine if a pre-occlusion condition is occurring and address the issue (e.g., slow pump motion down) before leak pressure is reached. Also the pump can be programmed to pause on every dispense stroke to obtain measured parameter readings, but doing so could have the undesirable effect of extending total delivery time when there is no occlusion.

As stated above, example embodiments of the occlusion detection algorithm are advantageous for significantly increasing occlusion detection reliability. The technical solution provided by the example embodiments of pausing the pump to increase the samples or sampling rate of a measured parameter indicative of pressure provides a much higher resolution for detecting an occlusion. Although this technical solution is described in connection with motor current sensing, it is not strictly confined to this implementation and can be used with any type of pressure sensing occlusion detection system. The pause also allows for the system to settle in order to make a higher number of more accurate measurements during a single stroke.

When a motor or drive train parameter measurement is implemented for pump operation, occlusion detection can be achieved by adding to the computer software instructions of the microcontroller 58, or a remote device that controls the medication delivery device 10, such operations as monitoring the parameter (e.g. motor current) and determining when a designated pressure correlated to the measured current meets a designated threshold(s). Thus, occlusion detection can be implemented via a software solution, and no hardware changes to the pump are needed.

It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network. Also, functional programs, codes, and code segments for accomplishing the illustrative embodiments can be easily construed as within the scope of claims exemplified by the illustrative embodiments by programmers skilled in the art to which the illustrative embodiments pertain. Method steps associated with the illustrative embodiments can be performed by one or more programmable processors executing a computer program, code or instructions to perform functions (e.g., by operating on input data and/or generating an output). Method steps can also be performed by, and apparatus of the illustrative embodiments can be implemented as, special purpose logic circuitry, e.g., an FPGA (field programmable gate array) or an ASIC (application-specific integrated circuit), for example.

The various illustrative logical blocks, modules, and circuits described in connection with the embodiments disclosed herein may be implemented or performed with a general purpose processor, a digital signal processor (DSP), an ASIC, a FPGA or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. Information carriers suitable for embodying computer program instructions and data include all forms of non-volatile memory, including by way of example, semiconductor memory devices, e.g., electrically programmable read-only memory or ROM (EPROM), electrically erasable programmable ROM (EEPROM), flash memory devices, and data storage disks (e.g., magnetic disks, internal hard disks, or removable disks, magneto-optical disks, and CD-ROM and DVD-ROM disks). The processor and the memory can be supplemented by, or incorporated in special purpose logic circuitry.

Those of skill in the art would understand that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof

Those of skill would further appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of claims exemplified by the illustrative embodiments. A software module may reside in random access memory (RAM), flash memory, ROM, EPROM, EEPROM, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. In other words, the processor and the storage medium may reside in an integrated circuit or be implemented as discrete components.

Computer-readable non-transitory media includes all types of computer readable media, including magnetic storage media, optical storage media, flash media and solid state storage media. It should be understood that software can be installed in and sold with a central processing unit (CPU) device. Alternatively, the software can be obtained and loaded into the CPU device, including obtaining the software through physical medium or distribution system, including, for example, from a server owned by the software creator or from a server not owned but used by the software creator. The software can be stored on a server for distribution over the Internet, for example.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. An infusion device comprising:
a pump comprising a chamber of fluid, and a pumping mechanism configured to control dispensing of a volume of fluid from the chamber during a dispense operation;
a pump measurement device configured to generate pump measurements indicative of pressure; and
a processing device configured to analyze one or more of the pump measurements obtained during a portion of the dispense operation, and pause the pump mechanism during the dispense operation when one or more of the pump measurements during the portion of the dispense operation satisfies a designated metric related to a designated pre-occlusion pressure.

2. The infusion device of claim 1, wherein the processing device is configured to control the pump mechanism to resume the dispense operation.

3. The infusion device of claim 2, wherein the processing device is configured to obtain another pump measurement during the dispense operation resumed after the pause, and to pause the pump mechanism again when the pump measurement satisfies a designated metric related to a designated pre-occlusion pressure.

4. The infusion device of claim 3, wherein the processing device is configured to control the pump mechanism to resume the dispense operation again.

5. The infusion device of claim 2, wherein the number of times the processing device pauses the dispense operation and resumes the dispense operation can be preconfigured, or dynamically determined, based on criteria chosen from pump motor current, pump motor voltage, encoder count, pump motor drive count, pump motor drive time, dispense operation energy, volume of the chamber, type of fluid in the chamber, and ambient air pressure.

6. The infusion device of claim 2, wherein the frequency with which the processing device pauses and resumes the dispense operation can be constant or vary throughout the dispense operation or within designated portions of the dispense operation.

7. The infusion device of claim 2, wherein the processing device is configured to perform a feedback loop of obtaining pump measurements and slowing down or speeding up the dispense operation based on the pump measurements.

8. The infusion device of claim 1, wherein the pump measurement is motor current, and the pump measurement device comprises a current sensing device configured to detect motor current of the pump during the dispense operation.

9. The infusion device of claim 1, wherein the pump is chosen from a positive displacement pump, and a syringe-style pump.

10. The infusion device of claim 1, wherein the designated metric relates to a designated pre-occlusion pressure is chosen from a range of measurements corresponding to pressures above normal pump operating pressures, and below a minimum leak pressure, and different from transient pressures related to pump start up or pump operation state change.

11. The infusion device of claim 1, wherein the processing device is configured to obtain at least an additional pump measurement during the pause, and controls the pump mechanism to resume the dispense operation when the additional pump measurement corresponds to a normal pump operating pressure and fails to satisfy the designated metric related to a designated pre-occlusion pressure.
